(19) United States
(12) Patent Application Publication
(10) Pub. No.: US 2015/0230859 A1
(43) Pub. Date: Aug. 20, 2015

(54) BI-DIRECTIONAL DEPLOYMENT OF NEUROMODULATION DEVICES AND ASSOCIATED SYSTEMS AND METHODS

(71) Applicant: Kevin Mauch, Windsor, CA (US)

(72) Inventor: Kevin Mauch, Windsor, CA (US)

(21) Appl. No.: 14/183,669

(22) Filed: Feb. 19, 2014

(51) Int. Cl. A61B 18/14

(52) U.S. Cl. CPC A61B 18/1492; A61B 2018/00434

(57) ABSTRACT

Methods for treating a patient using therapeutic renal neuromodulation and associated devices, systems, and methods are disclosed herein. One aspect of the present technology, for example, is directed to bi-directional control of helical- or spiral-shaped neuromodulation devices. A system can include, for example, a catheter having an elongated shaft and a treatment assembly at a distal portion of the elongated shaft. The catheter can further include a first control member configured to deploy a distal region of the treatment assembly and a second control member configured to deploy a proximal region of the treatment assembly. The proximal and distal regions of the treatment assembly are selectively transformable independent of each other.

### TECHNICAL FIELD

The present technology relates generally to devices and methods for deployment and positioning of neuromodulation devices. Some embodiments, for example, are directed to catheters, catheter systems, and methods for bi-directional control of helical/spiral neuromodulation devices.

### BACKGROUND

The sympathetic nervous system (SNS) is a primarily involuntary bodily control system typically associated with stress responses. SNS fibers that innervate tissue are present in almost every organ system of the human body and can affect characteristics such as pupil diameter, gut motility, and urinary output. Such regulation can have adaptive utility in maintaining homeostasis or preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiologies of hypertension, states of volume overload (such as heart failure), and progressive renal disease.

Sympathetic nerves innervating the kidneys terminate in the blood vessels, the juxtaglomerular apparatus, and the renal tubules. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone and likely contribute to increased blood pressure in hypertensive patients. For example, reduced renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology.

FIG. 1 is a partially-schematic perspective view of a neuromodulation system including a catheter configured in accordance with an embodiment of the present technology.

FIG. 2A is a side view of the catheter of FIG. 1 in a low-profile configuration in accordance with an embodiment of the present technology. A portion of the handle is removed for purposes of illustration.

FIG. 2B is a side view of the catheter of FIG. 1 in a deployed configuration in accordance with an embodiment of the present technology. A portion of the handle is removed for purposes of illustration.

FIG. 3 is a transverse cross-sectional view of a catheter support structure of FIG. 2A taken along the line 3-3. The adjacent control member is omitted for clarity.

FIG. 4 is a transverse cross-sectional view of the elongated shaft of the catheter of FIG. 2A taken along the line 4-4.

FIG. 5 is a side view of the treatment assembly in a distal deployed state in accordance with an embodiment of the present technology.

FIG. 6 is a side view of the treatment assembly in a proximal deployed state in accordance with an embodiment of the present technology.

FIG. 7A is a partial cross-sectional anatomical front view illustrating the catheter of FIG. 1 being advanced along an intravascular path in accordance with an embodiment of the present technology.

FIG. 7B is a side view, in partial longitudinal section, of a distal portion of a catheter with a treatment assembly in a low-profile or delivery state at a treatment site within a blood vessel in accordance with an embodiment of the present technology.

FIG. 7C is a side view of the treatment assembly, in partial longitudinal section, shown in a distal deployed state at a treatment site within a blood vessel in accordance with an embodiment of the present technology.

FIG. 7D is a side view of the treatment assembly, in partial longitudinal section, shown in a proximal deployed state at a treatment site within a blood vessel in accordance with an embodiment of the present technology.

FIG. 8 is a conceptual diagram illustrating the sympathetic nervous system and how the brain communicates with the body via the sympathetic nervous system.

FIG. 9 is an enlarged anatomical view illustrating nerves innervating a left kidney to form a renal plexus surrounding a left renal artery.

FIGS. 10A and 10B are anatomical and conceptual views, respectively, illustrating a human body including a brain and kidneys and neural efferent and afferent communication between the brain and kidneys.

FIGS. 11A and 11B are anatomic views illustrating, respectively, an arterial vasculature and a venous vasculature of a human.

### DETAILED DESCRIPTION

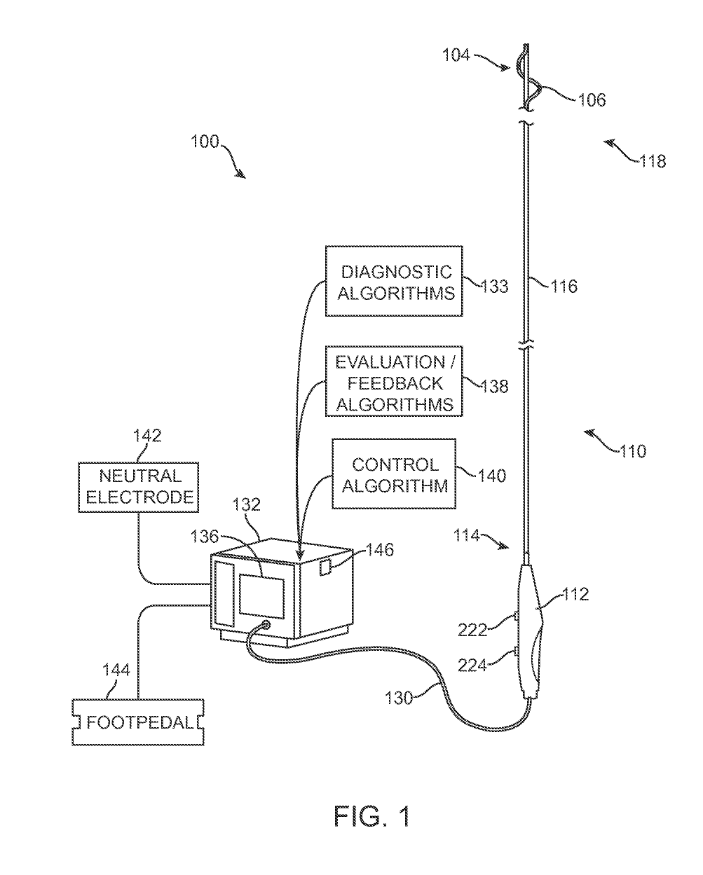

The present technology is directed to devices and methods for deployment and positioning of neuromodulation devices. Some embodiments of the present technology, for example, are directed to catheters, catheter systems, and methods for bi-directional control of helical/spiral neuromodulation devices. Specific details of several embodiments of the technology are described below with reference to FIGS. 1-11B. Although many of the embodiments are described below with respect to systems, devices, and methods for bi-directional control of helical/spiral neuromodulation devices, other applications (e.g., bi-directional control of non-helical or non-spiral neuromodulation devices, neuromodulation of other peripheral nerves, treatments other than neuromodulation, etc.) and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-11B.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

### I. SELECTED EMBODIMENTS OF NEUROMODULATION SYSTEMS

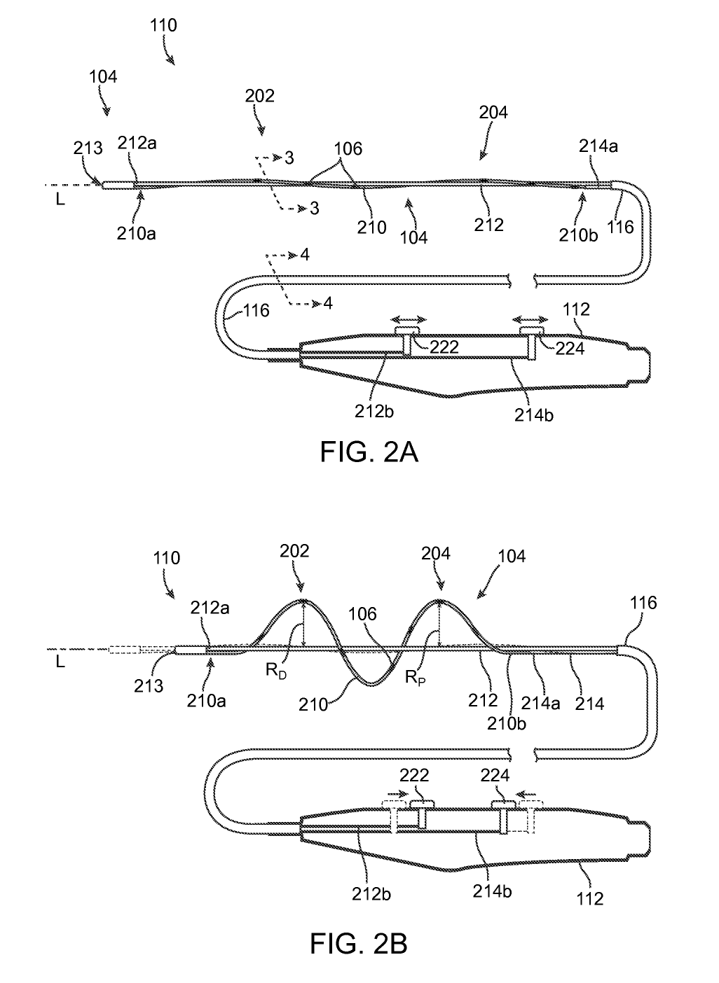

FIG. 1 is a partially-schematic perspective view of a neuromodulation system 100 (“system 100”) configured in accordance with an embodiment of the present technology. The system 100 includes an intravascular catheter 110 operably coupled to an energy source or energy generator 132 via a connector 130 (e.g., a cable). The catheter 110 can include an elongated shaft 116 having a proximal portion 114 and a distal portion 118. The catheter 110 also includes a handle assembly 112 at the proximal portion 114. The catheter 110 can further include a treatment assembly 104 carried by or affixed to the distal portion 118 of the elongated shaft 116. The treatment assembly 104 includes one or more energy delivery elements 106 (e.g., electrodes) configured to modulate nerves at or near the treatment location. The elongated shaft 116 is configured to intravascularly locate the treatment assembly 104 at a treatment location within a renal blood vessel or within another suitable body lumen (e.g., within a ureter) of a human patient (not shown). As described in greater detail below with reference to FIGS. 2A and 2B, the treatment assembly 104 may be deployed via a first actuator 222 and a second actuator 224, both actuators being carried by the handle 112.

The energy generator 132 can be configured to generate a selected form and/or magnitude of energy for delivery to the treatment site via the energy delivery element(s) 106 of the treatment assembly 104. For example, the energy generator 132 can be configured to generate RF energy (monopolar or bipolar), pulsed RF energy, microwave energy, ultrasound energy (e.g., intravascularly delivered ultrasound, extracorporeal ultrasound, high-intensity focused ultrasound (HIFU)), cryotherapeutic energy, direct heat energy, electromagnetic radiation (e.g., infrared, visible, gamma), or another suitable type of energy. In some embodiments, neuromodulation may be achieved by chemical-based treatment including delivering one or more chemicals (e.g., guanethidine, ethanol, phenol, a neurotoxin (e.g., vincristine)), or another suitable agent selected to alter, damage, or disrupt nerves. Furthermore, the energy generator 132 can be configured to control, monitor, supply, or otherwise support operation of the catheter 110. For example, a control mechanism, such as foot pedal 144, may be connected (e.g., pneumatically connected or electrically connected) to the energy generator 132 to allow an operator to initiate, terminate and/or adjust various operational characteristics of the energy generator, such as power delivery. In some embodiments, the energy generator 132 may be configured to provide delivery of a monopolar electric field via the energy delivery element(s) 106. In such embodiments, a neutral or dispersive electrode 142 may be electrically connected to the energy generator 132 and attached to the exterior of the patient (not shown). Although the following description of the technology specifies an embodiment that delivers RF energy such that energy delivery element(s) 106 are electrodes, it will be understood by those of skill in the art that delivering some of the alternate non-electrical treatments mentioned above would require adapting the system, e.g. using optical fibers or hollow tubing instead of wires.

In some embodiments, the system 100 includes a remote control device (not shown) that can be configured to be sterilized to facilitate its use within a sterile field. The remote control device can be configured to control operation of the treatment assembly 104, the energy generator 132, and/or other suitable components of the system 100. For example, the remote control device can be configured to allow for selective activation of the energy delivery elements 106. In other embodiments, the remote control device may be omitted and its functionality may be incorporated into the handle 112 or energy generator 132.

As shown in FIG. 1, the energy generator 132 can further include an indicator or display screen 136. The energy generator 132 can include other indicators, including one or more LEDs, a device configured to produce an audible indication, and/or other suitable communicative devices. In the embodiment shown in FIG. 1, the display 136 includes a user interface configured to receive information or instructions from a user and/or provide feedback to the user. For example, the energy generator 132 can be configured to provide feedback to an operator before, during, and/or after a treatment procedure via the display 136. The feedback can be based on output from one or sensors (not shown) associated with the treatment assembly 104 such as temperature sensor(s), impedance sensor(s), current sensor(s), voltage sensor(s), flow sensor(s), chemical sensor(s), ultrasound sensor(s), optical sensor(s), pressure sensor(s) and/or other sensing devices.

The system 100 can further include a controller 146 having, for example, memory (not shown) and processing circuitry (not shown). The memory and storage devices are computer-readable storage media that may be encoded with non-transitory, computer-executable instructions such as diagnostic algorithm(s) 133, evaluation/feedback algorithm(s) 138, and/or control algorithm(s) 140. The control algorithms 140 can be executed on a processor (not shown) of the system 100 to control energy delivery to the energy delivery element(s) 106. In some embodiments, selection of one or more parameters of an automated control algorithm 140 for a particular patient may be guided by diagnostic algorithm(s) 133 that measure and evaluate one or more operating parameters prior to energy delivery. The diagnostic algorithm(s) 133 provide patient-specific feedback to the clinician prior to activating the energy delivery element(s) 106 that can be used to select an appropriate control algorithm 140 and/or modify the control algorithm 140 to increase the likelihood of efficacious neuromodulation.

Although the controller 146 is incorporated into the energy generator 132 in the embodiment shown in FIG. 1, in other embodiments the controller 146 may be an entity distinct from the energy generator 132. For example, additionally or alternatively, the controller 146 can be a personal computer(s), server computer(s), handheld or laptop device(s), multiprocessor system(s), microprocessor-based system(s), programmable consumer electronic(s), digital camera(s), network PC(s), minicomputer(s), mainframe computer(s), and/or any suitable computing environment.

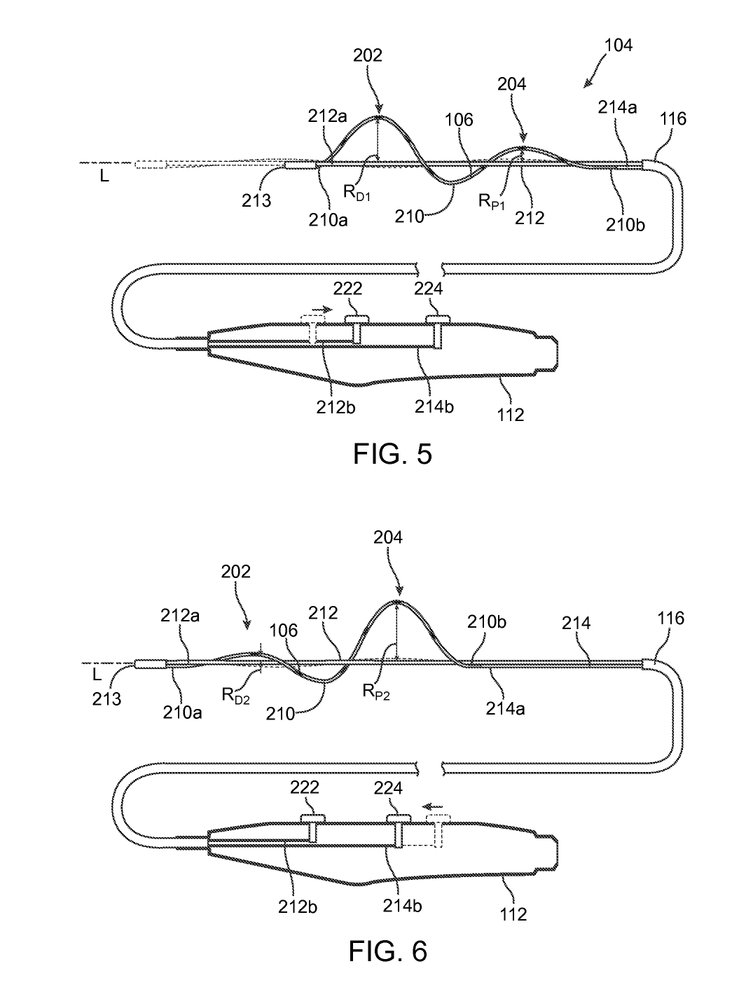

FIG. 2A is a side view of the catheter 110 with the treatment assembly 104 in a low-profile or delivery state. A portion of the handle 112 is removed for illustrative purposes. As shown, the treatment assembly 104 can include a flexible, tubular support structure 210 having a distal region 202 and a proximal region 204. The support structure 210 is configured to carry the energy delivery elements 106. FIG. 2B is a side view illustrating the catheter 110 with the treatment assembly 104 in a deployed configuration. As shown in FIG. 2B, the support structure 210 can comprise a generally helical/spiral shape when in the deployed configuration. As used herein, “deployed” refers to the configuration of the support structure 210 when at least one of (a) a radial dimension R_{D} between the distal region 202 of the support structure 210 and a longitudinal axis L of the shaft 116 and (b) a radial dimension R_{P} between the proximal region 204 and the longitudinal axis L of the shaft 116, is greater than the radial dimension between the respective region and the longitudinal axis L when the support structure 210 is in the low-profile or delivery configuration (FIG. 2A). As discussed in greater detail below with reference to FIGS. 5 and 6, the distal and proximal regions 202, 204 of the support structure 210 can be deployed independently of one another so that the radial dimension R_{D} at the distal region can be greater than, less than, or generally the same as the radial dimension R_{P} at the proximal region.

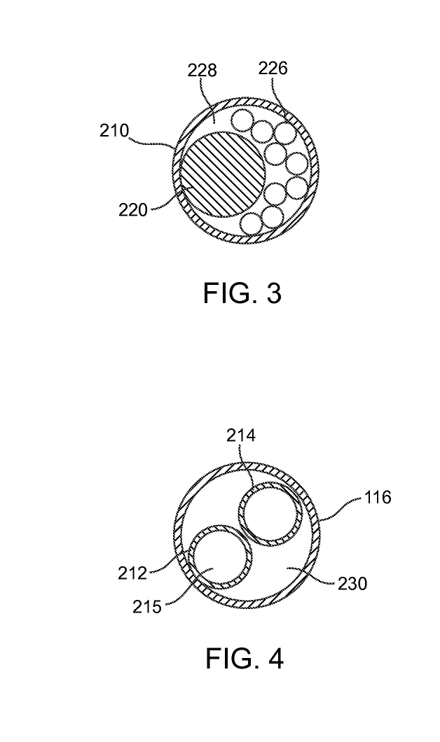

FIG. 3 is a transverse cross-sectional view of the support structure 210 taken along line 3-3 of FIG. 2A. Adjacent control member 212 is omitted for clarity. As best seen in FIG. 3, the support structure 210 can be a flexible tube and the treatment assembly 104 (FIG. 2A) can include a pre-shaped member 220 positioned within a lumen 228 of the tube. The support structure 210 can be configured to fit tightly against the member 220 and/or wires 226 to reduce space between an inner portion of the support structure 210 and the components positioned therein. For example, the member 220 and the inner wall of the support structure 210 can be in intimate contact such that there is little or no space between the member 220 and the support structure 210. Such an arrangement can help to reduce or prevent the formation of wrinkles in the support structure 210 and/or treatment assembly 104 (FIG. 2A) during deployment. The support structure 210 may be composed of a polymer material such as polyamide, polyimide, polyether block amide copolymer sold under the trademark PEBAX, polyethylene terephthalate (PET), a polyether ether ketone (PEEK) polymer, polypropylene, thermoplastic polyurethanes (TPUs) such as an aliphatic, polycarbonate-based TPU sold under the trademark CARBOTHANE, or an aromatic polyether-based TPU sold under the trademark ELATHANE, or another suitable material that provides sufficient flexibility to the support structure 210.

Upon deployment, the pre-shaped member 220 can bias at least a portion of the support structure 210 into a deployed state. For example, the member 220 can have a pre-set configuration that tends to give at least a portion of the support structure 210 a helical/spiral configuration in the deployed state. When deployed, the helical/spiral shape can define a virtual cylinder having a constant diameter (FIG. 2B), or a virtual cone that tapers in a proximal direction along all or part of the length of the support structure 210 (FIG. 5), or a virtual cone that tapers in a distal direction along all or part of the length of the support structure 210 (FIG. 6). The helical/spiral shape can include one or more full or partial turns around the circumference of the virtual cylinder or cone, and the number of turns is not limited to the configuration illustrated in the figures. The member 220 may be formed from suitable elastic, pseudo-elastic or superelastic materials, e.g., spring-temper stainless steel or nickel-titanium alloy (nitinol) that are pre-formed or pre-shaped into the desired deployed state. In other embodiments, the member 220 may be composed of different materials and/or have a different configuration (not shown). For example, the member 220 can include a tubular structure comprising a nitinol multifilar stranded wire with a lumen therethrough and sold under the trademark HELICAL HOLLOW STRAND (HHS) (commercially available from Fort Wayne Metals of Fort Wayne, Ind.). Alternatively, the member 220 may be formed from multiple materials such as a composite of one or more polymers and metals.

Referring again to FIGS. 2A and 2B together, the energy delivery elements or electrodes 106 can be separate band electrodes axially spaced apart along the support structure 210. In the illustrated embodiment, for example, the electrodes 106 are adhesively bonded to the support structure 210 at different positions along the length of the support structure 210. In some embodiments, the electrodes 106 are formed from a suitable electrically conductive material (e.g., a metal, such as gold, platinum, alloys of platinum and iridium, etc.). The number, arrangement, shape, and/or composition of the electrodes 106 may vary. The individual electrodes 106 can be electrically connected to the energy generator 132 (FIG. 1) by conductors or bifilar wires 226 extending through a lumen 228 of the support structure 210 and/or shaft 116 (FIG. 3). For example, the individual energy delivery elements 106 may be welded or otherwise electrically coupled to corresponding wires 226, and the wires 226 can extend through the elongated shaft 116 for the entire length of the shaft 116 such that proximal ends of the wires 226 are coupled to the handle 112 and/or to the energy generator 132.

As mentioned previously, the handle 112 includes first actuator 222 and second actuator 224. The first actuator 222, for example, is movable between a first initial position (FIG. 2A) and a first deployed position (FIG. 2B) that is proximal to the first initial position along the length of the handle 112. The second actuator 224 is movable, independently of the first actuator 222, between a second initial position (FIG. 2A) and a second deployed position (FIG. 2B) that is distal to the second initial position along the length of the handle 112. As shown in FIG. 2A, when both the first actuator 222 and the second actuator 224 are in the respective initial positions, the support structure 210 is in the low-profile configuration.

FIG. 4 is a transverse cross-sectional view of the elongated shaft 116 taken along line 4-4 in FIG. 2A. Referring to FIGS. 2A-4 together, the catheter 110 can further include a first elongated control member 212 slidably extending through shaft 116 and treatment assembly 104 to operatively connect a distal end 210a of the support structure 210 to the first actuator 222. First control member 212 can have a lumen 215 extending proximally from an opening 213 at the distal end of the first control member 212 to the handle 112. In some embodiments, proximal portion 212b of first control member 212 can extend to or beyond the proximal end of handle 112 and lumen 215 can be configured to receive a guidewire (not shown) therethrough from proximal portion 212b to opening 213. In other embodiments, the first control member 212 can be a solid structure (e.g., a wire, a rod, multifilar cable, etc.).

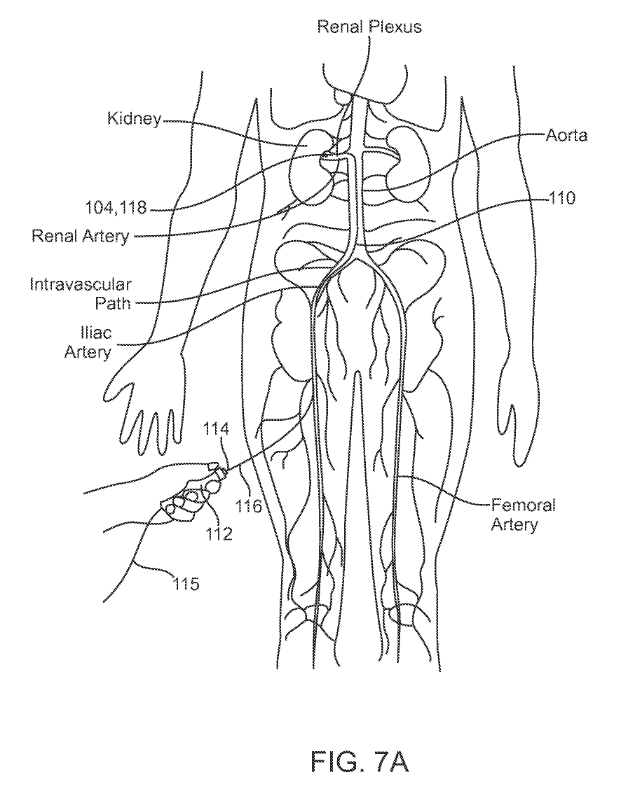

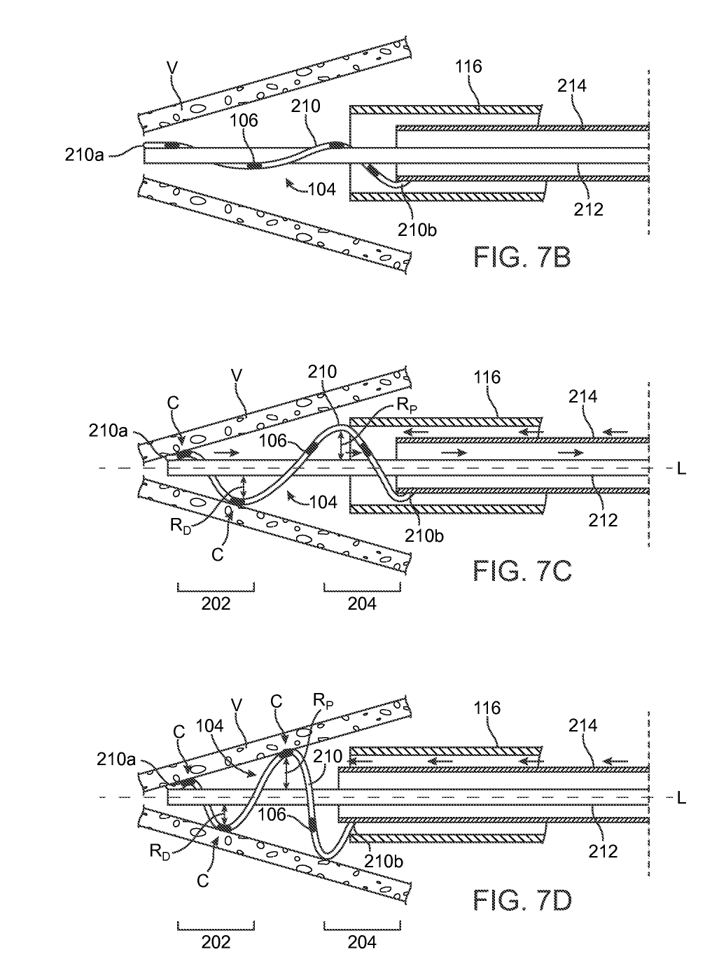

The catheter 110 can also include a second elongated tubular control member 214 slidably extending through shaft 116 to operatively connect the proximal end 210b of the support structure 210 to the second actuator 224. In this embodiment, support structure 210 may be a continuation of control member 214 such that pre-shaped member 220 and wires 226 may extend through both structure 210 and member 214. In other embodiments, the second control member 214 can be a solid structure (e.g., a wire, rod, etc.). As shown in FIG. 4, the first control member 212 and the second control member 214 can be positioned proximate one another within a central lumen 230 of the shaft 116. In other embodiments, however, the first control member 212 and the second control member 214 can have other suitable shapes, sizes and/or arrangements. In some embodiments, for example, the first control member 212 may be slidably positioned within the second control member 214 while the second control member 214 remains slidably positioned within the shaft 116 (see, e.g., FIGS. 7B-7D). In this coaxial embodiment, wires 226 may extend through central lumen 230 alongside control member 214.

As best shown in FIGS. 2A and 2B, in operation, longitudinal movement of the first actuator 222 in a proximal direction causes proximal longitudinal movement of the first control member 212 with respect to both shaft 116 and second control member 214. This proximal longitudinal movement of the first control member 212 pulls the distal end 210a of the support structure 210 proximally, which in turn shortens the length of treatment region 104 and increases a radial dimension R_{D} between the distal region 202 of the support structure 210 and the longitudinal axis L of the shaft 116. Radial dimension R_{D} may be considered as being measured directly to a portion of support structure 210, or to a virtual surface of a cylinder or cone defined by the support structure. Similarly, longitudinal movement of the first actuator 222 in a distal direction (not shown) causes distal longitudinal movement of the first control member 212 with respect to both shaft 116 and second control member 214. This distal longitudinal movement of the first control member 212 pushes the distal end 210a of the support structure 210 distally, which in turn increases the length of treatment region 104 and decreases radial dimension R_{D} between the distal region 202 of the support structure 210 and the longitudinal axis L of the shaft 116.

With respect to the second actuator 224, in operation, longitudinal movement of the second actuator 224 in a distal direction causes distal longitudinal movement of the second control member 214 with respect to both shaft 116 and first control member 212. This distal longitudinal movement of the second control member 214 pushes the proximal end 210b of the support structure 210 distally, which in turn shortens the length of treatment region 104 and increases a radial dimension R_{P} between the proximal region 204 of the support structure 210 and the longitudinal axis L of the shaft 116. Radial dimension R_{P} may be considered as being measured directly to a portion of support structure 210, or to a virtual surface of a cylinder or cone defined by the support structure. Similarly, proximal longitudinal movement of the second actuator 224 causes proximal longitudinal movement of the second control member 214 with respect to both shaft 116 and first control member 212. This proximal longitudinal movement of the second control member 214 pulls the proximal end 210b of the support structure 210 proximally, which in turn increases the length of treatment region 104 and decreases a radial dimension R_{P} between the proximal region 204 of the support structure 210 and the longitudinal axis L of the shaft 116.

In the present technology, proximal and/or distal movement of the first actuator 222 is independent of proximal and/or distal movement of the second actuator 224. Likewise, proximal and/or distal movement of the first control member 212 and/or distal end 210a of the support structure 210 are independent of proximal and/or distal movement of the second control member 214 and/or proximal end 210b of the support structure 210. As a result, the distal end 210a of the support structure 210 can be moved proximally and/or distally while the proximal end 210b of the support structure 210 and/or the shaft 116 remain generally stationary (in a low-profile or deployed position) relative to the patient. FIG. 5, for example, shows the treatment assembly 104 in a “distal-deployed configuration,” that is, the configuration of the support structure 210 when the distal region 202 of the support structure 210 has been radially deployed towards or against the inner wall of a target vessel more than the proximal region 204 of the support structure 210 (R_{D1}>R_{P1}). The vessel wall is omitted in FIG. 5 for clarity, but see also FIG. 7C. Likewise, the proximal end 210b of the support structure 210 can be moved proximally and/or distally while the distal end 210a of the support structure remains generally stationary relative to the patient. For example, FIG. 6 shows the treatment assembly 104 in a “proximal-deployed configuration,” that is, the configuration of the support structure 210 when the proximal region 204 of the support structure 210 has been radially deployed towards or against the inner wall of a target vessel more than the distal region 202 of the support structure 210 (R_{P2}>R_{D2}). The vessel wall is omitted in FIG. 6 for clarity, but see also FIG. 7D. In some embodiments, the distal and proximal ends 210a, 210b of the support structure 210 may be moved in a distal direction concurrently, in a proximal direction concurrently, or in opposite directions concurrently.

### II. SELECTED DELIVERY EMBODIMENTS

FIGS. 7A-7D (with additional reference to FIG. 1) illustrates at least one step of modulating renal nerves with an embodiment of the system 100. In FIG. 7A, intravascular delivery of the catheter 110 can include percutaneously inserting a guide wire 115 within the vasculature at an access site (e.g., femoral (illustrated), brachial, radial, or axillary artery) and moving the shaft 116 and the treatment assembly 104 (in the delivery state) along the guide wire 115 until at least a portion of the treatment assembly 104 reaches the treatment location (as shown in FIG. 7A). In other embodiments, the distal portion 118 may be delivered to the treatment site within a guide sheath (not shown) with or without using the guide wire 115. In still other embodiments, the shaft 116 may be steerable itself such that the treatment assembly 104 at the distal portion 118 may be delivered to the treatment site without the aid of the guide wire 115 and/or guide sheath.

Image guidance, e.g., CT, fluoroscopy, IVUS, OCT, ultrasonography, or another suitable guidance modality, or combinations thereof, may be used to aid the clinician's positioning and manipulation of the distal portion 118 and/or the treatment assembly 104. For example, a fluoroscopy system (e.g., including a flat-panel detector, x-ray, or c-arm) can be rotated to accurately visualize and identify the target treatment site. In other embodiments, the treatment site can be located using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the catheter 110 (FIG. 1). Further, in some embodiments, image guidance components (e.g., IVUS, OCT) may be integrated with the catheter 110 and/or run in parallel with the catheter 110 to provide image guidance during positioning of the treatment assembly 104. For example, image guidance components (e.g., IVUS or OCT) can be coupled to a distal portion of the catheter 110 to provide three-dimensional images of the vasculature proximate the target site to facilitate positioning or deploying the treatment assembly 104 within the target renal blood vessel.

FIG. 7B is a side view, in partial longitudinal section, of a distal portion of catheter 110 with treatment assembly 104 in a low-profile or delivery state at a treatment site within a renal artery such as vessel V. FIGS. 7C and 7D, using views similar to FIG. 7A, illustrate the treatment assembly 104 in different deployed states. As shown in FIG. 7C, once the treatment assembly 104 is positioned at a treatment location, the first control member 212 can be pulled proximally, via the first actuator 222 (FIG. 2B), to increase a radial dimension R_{D} at the distal region 202 of the support structure 210 and bring at least the electrodes 106 at or near the distal region 202 in contact C with vessel V. As shown in FIG. 7D, the second control member 224 can then be pushed distally, via the second actuator 224 (FIG. 2B), to increase the radial dimension R_{P} at the proximal region 204 and bring the electrodes 106 at or near the proximal region 204 in contact C with the vessel V. In other embodiments, the distal and proximal regions 202, 204 can be deployed in any order and/or simultaneously.

Depending on the morphology of the vessel V at the treatment site, the distal and proximal regions 202, 204 of the support structure 210 may be selectively increased and/or decreased (continuously or incrementally) to the same or different radial dimensions R_{D}, R_{P} (e.g., R_{D}>Rₚ, R_{D}<Rₚ, or R_{D}=R_{P}). For example, when the present technology is applied to a tapered vessel (e.g., a tapered renal artery) the first and second actuators 222, 224 (FIG. 2B) can be manipulated so that a radial dimension between the longitudinal axis and the elongated shaft selectively decreases in a direction of the tapering of the vessel such that the treatment section is adapted to bring the electrodes at both the proximal region and the distal region of the treatment section into apposition with an inner wall of the vessel. In these and other embodiments, the treatment assembly 104 can be configured to adapt to other vessel morphologies besides tapered blood vessels, such as any vessel or portion of a vessel with a tortuous or and/or unpredictable morphology.

Once positioned and deployed, the electrodes 106 can be activated to modulate the nerves (not shown) proximate to the wall of vessel V. For example, the treatment assembly 104 can be configured to form a lesion or series of lesions (e.g., a helical/spiral lesion or a discontinuous lesion pattern) that is fully-circumferential overall, but non-circumferential in a plane normal to the vessel axis at any of the treatment locations. This can facilitate precise and efficient treatment with a low possibility of vessel stenosis. In other embodiments, the treatment assembly 104 can be configured to form a partially-circumferential lesion or a fully-circumferential lesion at a single longitudinal segment of the treatment location.

The radial dimensions R_{D}, R_{P} at the distal and/or proximal regions can be adjusted (e.g., increased and/or decreased) any number of times and at any point before, during, and/or after electrode activation to improve electrode 106 contact with the vessel wall V. For example, the treatment assembly 104 can include one or more sensors (not shown) that can continuously or intermittently monitor various parameters (e.g., impedance, change in impedance, temperature, change in temperature, etc.) associated with the sensors and/or the electrodes 106 and/or the tissue at or near the treatment site. For example, an impedance measurement can be made based on a monopolar electric circuit that includes an electrode 106 or a sensor and neutral electrode 142. Alternatively, an impedance measurement can be made based on a bipolar electric circuit that includes a pair of electrodes 106 or sensors. Based on such monitored parameters, the clinician may decide to reposition the treatment assembly 104 and re-activate the electrodes 106. For example, repositioning can include decreasing and/or increasing the radial dimension at the distal and/or proximal region R_{D}, R_{P}, longitudinally advancing or retracting the treatment assembly 104 along the longitudinal axis L and/or rotating the treatment assembly 104 about the longitudinal axis L.

### III. NEUROMODULATION

Neuromodulation is the partial or complete incapacitation or other effective disruption of nerves innervating, for example, an organ. As an example, renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and, in particular, conditions associated with central sympathetic overstimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, osteoporosis, and sudden death, among others. The reduction of afferent neural signals typically contributes to the systemic reduction of sympathetic tone/drive, and renal neuromodulation is expected to be useful in treating several conditions associated with systemic sympathetic overactivity or hyperactivity. Renal neuromodulation can potentially benefit a variety of organs and bodily structures innervated by sympathetic nerves.

Thermal effects can include both thermal ablation and non-ablative thermal alteration or damage (e.g., via sustained heating and/or resistive heating) to partially or completely disrupt the ability of a nerve to transmit a signal. Desired thermal heating effects, for example, may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for ablative thermal alteration. More specifically, exposure to thermal energy in excess of a body temperature of about 37° C., but below a temperature of about 45° C., may induce thermal alteration via moderate heating of target neural fibers or of vascular structures that perfuse the target fibers. In cases where vascular structures are affected, the target neural fibers may be denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures that perfuse the target fibers. In some patients, it may be desirable to achieve temperatures that thermally ablate the target neural fibers or the vascular structures, but that are less than about 90° C., or less than about 85° C., or less than about 80° C., and/or less than about 75° C. Other embodiments can include heating tissue to a variety of other suitable temperatures. Regardless of the type of heat exposure utilized to induce the thermal neuromodulation, a reduction in renal sympathetic nerve activity (RSNA) is expected.

Various techniques can be used to partially or completely incapacitate neural pathways, such as those innervating the kidneys. The purposeful application of energy (e.g., RF energy, mechanical energy, acoustic energy, electrical energy, thermal energy, etc.) to tissue and/or the purposeful removal of energy (e.g., thermal energy) from tissue can induce one or more desired thermal heating and/or cooling effects on localized regions of the tissue. The tissue, for example, can be tissue of the renal artery and adjacent regions of the renal plexus, which lay intimately within or adjacent to the adventitia of the renal artery. For example, the purposeful application and/or removal of energy can be used to achieve therapeutically effective neuromodulation along all or a portion of the renal plexus.

Many current helical or spiral neuromodulation systems deploy mainly from a distal to proximal direction. Often times the distal end of the electrode array is movable in a proximal and distal direction but the proximal end of the electrode array is fixed relative to the catheter shaft. As a result, in vessels or portions of vessels with tapered diameters (e.g., the renal artery), many devices first deploy distally where the vessel diameter is smaller, which can prevent the electrodes at the proximal end of the helical structure from making contact with the inner vessel wall. Likewise, achieving contact with the vessel wall along a substantial length of the helical or spiral device can be difficult in vessels with tortuous or unpredictable morphologies. To address this need, the present technology provides several embodiments of devices, systems, and methods that provide bi-directional deployment of a helical or spiral device to better position the electrodes in apposition with the vessel wall.

### IV. PERTINENT ANATOMY AND PHYSIOLOGY

The following discussion provides further details regarding pertinent patient anatomy and physiology. This section is intended to supplement and expand upon the previous discussion regarding the relevant anatomy and physiology, and to provide additional context regarding the disclosed technology and the therapeutic benefits associated with renal neuromodulation. For example, as mentioned previously, several properties of the renal vasculature may inform the design of catheters and associated methods for achieving renal neuromodulation, and impose specific design requirements for such devices. Specific design requirements may include accessing the renal artery, ureter, or renal pelvic anatomy, facilitating stable contact between a therapeutic element of a catheter and a luminal surface or wall, and/or effectively modulating the renal nerves using the therapeutic element.

A. The Sympathetic Nervous System

The SNS is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS extend through tissues in almost every organ system, providing at least some regulatory function to characteristics as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

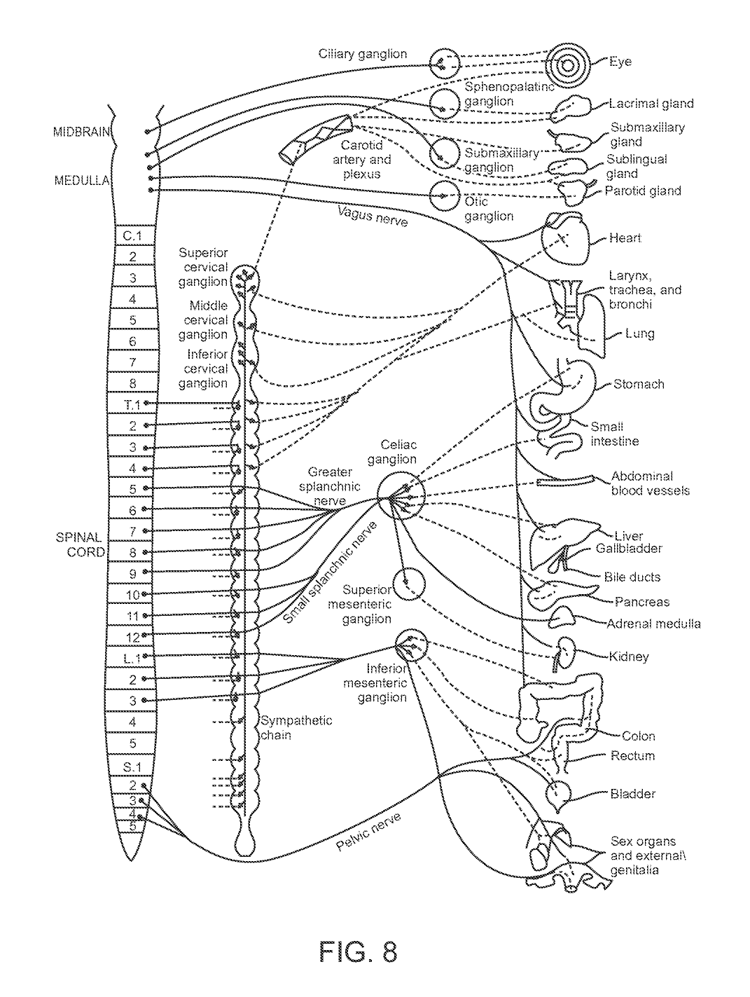

As shown in FIG. 8, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which send sympathetic nerve fibers to the head and thorax organs, and the celiac and mesenteric ganglia (which send sympathetic fibers to the gut).

2. Nerves of the Kidneys

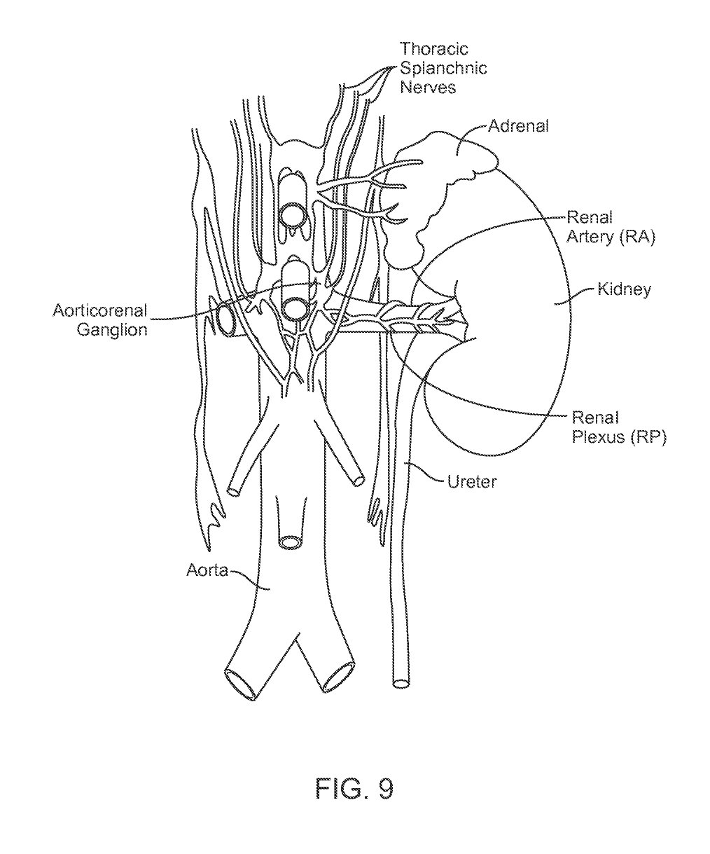

As shown in FIG. 9, the kidney neural system includes the renal plexus, which is intimately associated with the renal artery. The renal plexus is an autonomic plexus that surrounds the renal artery and is embedded within the adventitia of the renal artery. The renal plexus extends along the renal artery until it arrives at the substance of the kidney. Fibers contributing to the renal plexus arise from the celiac ganglion, the superior mesenteric ganglion, the aorticorenal ganglion and the aortic plexus. The renal plexus, also referred to as the renal nerve, is predominantly comprised of sympathetic components. There is no (or at least very minimal) parasympathetic neural activity of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

i. Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

ii. Renal Sensory Afferent Nerve Activity

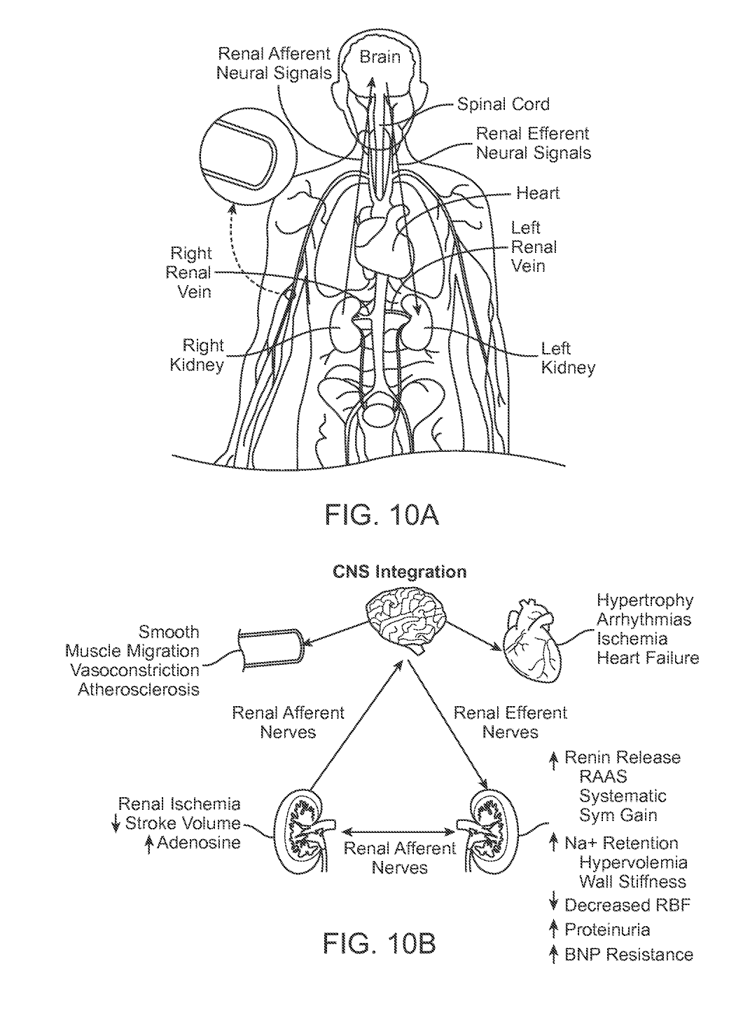

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine may trigger activation of afferent neural communication. As shown in FIGS. 10A and 10B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, fluid volume retention, and vasoconstriction. Central sympathetic over activity also impacts other organs and bodily structures having sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, sodium retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal neuromodulation, a desirable reduction of central sympathetic outflow to various other organs such as the heart and the vasculature is anticipated.

B. Additional Clinical Benefits of Renal Neuromodulation

As provided above, renal neuromodulation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal neuromodulation might also be useful in treating other conditions associated with systemic sympathetic hyperactivity. Accordingly, renal neuromodulation may also benefit other organs and bodily structures having sympathetic nerves, including those identified in FIG. 8.

C. Achieving Intravascular Access to the Renal Artery

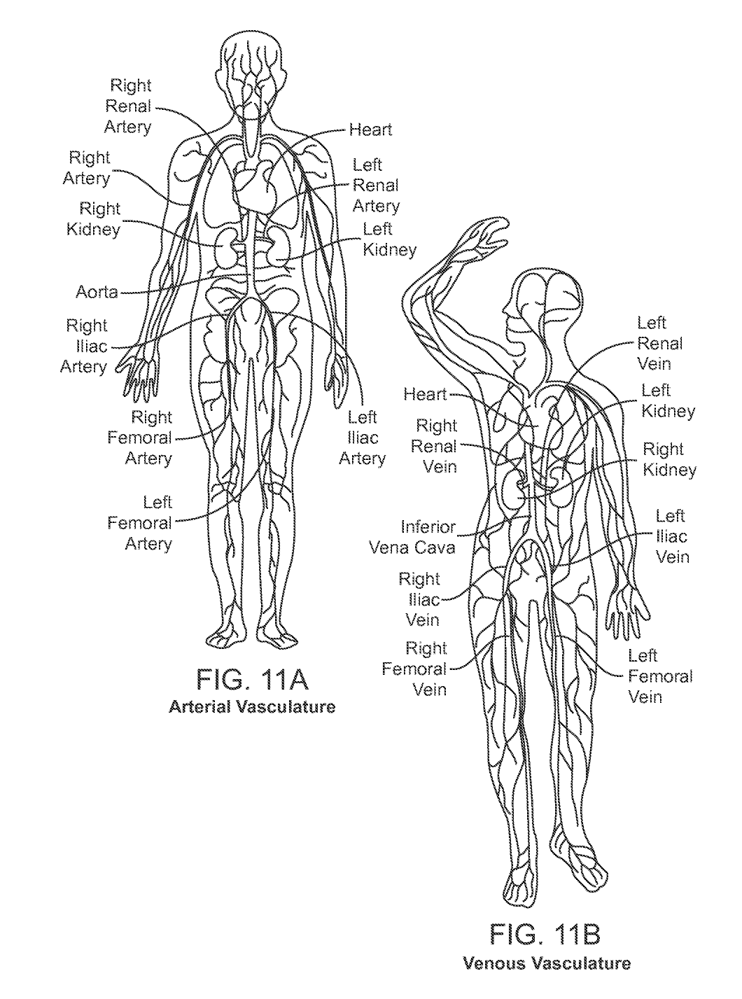

In accordance with the present technology, neuromodulation of a left and/or right renal plexus RP, which is intimately associated with a left and/or right renal artery, may be achieved through intravascular access. As FIG. 11A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 11B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, consistent positioning and appropriate contact force applied by the energy delivery element to the vessel wall can be important for predictability. However, navigation typically is impeded by the tight space within a renal artery, as well as tortuosity of the artery. Furthermore, establishing consistent contact can be complicated by patient movement, respiration, and/or the cardiac cycle. These factors, for example, may cause significant movement of the renal artery relative to the aorta, and the cardiac cycle may transiently distend the renal artery (i.e., cause the wall of the artery to pulse).

After accessing a renal artery and facilitating stable contact between neuromodulatory apparatus and a luminal surface of the artery, nerves in and around the adventitia of the artery can be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy can be delivered to the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Accordingly, the complex fluid mechanics and thermodynamic conditions present in the renal artery during treatment, particularly those that may impact heat transfer dynamics at the treatment site, may be important in applying energy from within the renal artery.

The neuromodulatory apparatus can be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

Blood flow through a renal artery may be temporarily occluded for a short time with minimal or no complications. However, occlusion for a significant amount of time can be avoided in some cases to reduce the likelihood of injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the take-off angle of a renal artery relative to the aorta. These properties will be discussed in greater detail with respect to the renal arteries. However, depending on the apparatus, systems, and methods utilized to achieve renal neuromodulation, such properties of the renal arteries also may guide and/or constrain design characteristics.

As noted above, an apparatus positioned within a renal artery can conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment can be important to reach the target neural fibers, the treatment can be prevented from becoming too deep (e.g., >5 mm from inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, which located at the distal end of the renal artery, may move as much as 4 inches cranially with respiratory excursion. This may impart significant motion to the renal artery connecting the aorta and the kidney, thereby requiring from the neuromodulatory apparatus a unique balance of stiffness and flexibility to maintain contact between the thermal treatment element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### V. CONCLUSION

The above detailed descriptions of embodiments of the present technology are for purposes of illustration only and are not intended to be exhaustive or to limit the present technology to the precise form(s) disclosed above. Various equivalent modifications are possible within the scope of the present technology, as those skilled in the relevant art will recognize For example, while steps may be presented in a given order, alternative embodiments may perform steps in a different order. The various embodiments described herein and elements thereof may also be combined to provide further embodiments. In some cases, well-known structures and functions have not been shown or described in detail to avoid unnecessarily obscuring the description of embodiments of the present technology.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Certain aspects of the present technology may take the form of computer-executable instructions, including routines executed by a controller or other data processor. In some embodiments, a controller or other data processor is specifically programmed, configured, and/or constructed to perform one or more of these computer-executable instructions. Furthermore, some aspects of the present technology may take the form of data (e.g., non-transitory data) stored or distributed on computer-readable media, including magnetic or optically readable and/or removable computer discs as well as media distributed electronically over networks. Accordingly, data structures and transmissions of data particular to aspects of the present technology are encompassed within the scope of the present technology. The present technology also encompasses methods of both programming computer-readable media to perform particular steps and executing the steps.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A catheter apparatus, comprising:
an elongated tubular shaft having a proximal portion and a distal portion;
a treatment assembly at the distal portion of the shaft and configured to be located at a target location within a blood vessel of a human patient, wherein the treatment assembly includes a distal region, a proximal region, and a plurality of energy delivery elements;
a handle at the proximal portion of the shaft;
a first elongated control member slidably positioned within the shaft and operatively connected between the distal region of the treatment assembly and a first actuator carried by the handle;
a second elongated control member slidably positioned within the shaft and operatively connected between the proximal region of the treatment assembly and a second actuator carried by the handle;
wherein
the distal region of the treatment assembly is transformable between a low-profile delivery configuration and a first deployed configuration via actuation of the first control member;
the proximal region of the treatment assembly is transformable, independent of the distal region, between the low-profile delivery configuration and a second deployed configuration via actuation of the second control member;
the treatment assembly comprises a helical shape when the distal and proximal regions of the treatment assembly are in the first and second deployed configurations, respectively.

2. The catheter apparatus of claim 1 wherein:
longitudinal movement of the first actuator in a proximal direction causes proximal longitudinal movement of the first elongated member; and
longitudinal movement of the second actuator in a distal direction causes distal longitudinal movement of the second elongated member.

3. The catheter apparatus of claim 1 wherein the plurality of energy delivery elements comprise one or more first energy delivery elements along the distal region and one or more second energy elements along the proximal region, and wherein:
when the distal region of the treatment assembly is in the first deployed configuration, the one or more first energy delivery elements at the distal region are configured to be in apposition with an inner wall of the blood vessel, and
when the proximal region of the treatment assembly is in the second deployed configuration, the one or more second energy delivery elements at the proximal region are configured to be in apposition with the inner wall of the blood vessel.

4. The catheter apparatus of claim 1 wherein the first control member comprises a first flexible control rod and the second control member comprises a second flexible control rod.

5. The catheter apparatus of claim 1 wherein the distal and proximal regions of the treatment assembly are configured to be transformed concurrently.

6. A catheter apparatus for placement in a blood vessel of a human patient, the catheter apparatus comprising:
an elongated shaft extending along a longitudinal axis, wherein the elongated shaft includes a proximal portion and a distal portion, and wherein the distal portion is configured for intraluminal delivery to the blood vessel;
a treatment section carried by the distal portion of the shaft, the treatment section including a distal region and a proximal region, and wherein the distal region and the proximal region of the treatment section are configured to independently move proximally and/or distally relative to the elongated shaft and also relative to each other; and
a plurality of energy delivery elements carried by the treatment section and configured to delivery radio frequency (RF) energy across an interior wall of the blood vessel to nerves along the blood vessel,
wherein the treatment section is transformable within the blood vessel between a low-profile delivery configuration and a deployed configuration having a spiral shape,
wherein, in the deployed configuration, a radial dimension between the treatment section and the longitudinal axis selectively decreases in a proximal and/or distal direction such that the treatment section is adapted to bring the energy delivery elements at the proximal and distal regions of the treatment section in contact with an inner wall of the blood vessel.

7. The catheter apparatus of claim 6, further comprising:
a control device connected to the proximal portion of the shaft and configured to be positioned external of the patient while the distal shaft portion is within the blood vessel, wherein the control portion includes a first control member and a second control member;
a first elongated member extending between a distal end of the treatment section and the first control member; and
a second elongated member extending between a proximal end of the treatment section and the second control member,
wherein proximal movement of the first control member causes an increase in a first radial dimension between the distal region of the treatment section and the longitudinal axis, and
wherein distal movement of the second control member causes an increase in a second radial dimension between the proximal region of the treatment section and the longitudinal axis.

8. A method, comprising:
intravascularly positioning a catheter at a treatment site within a blood vessel of a human patient, wherein the intravascular catheter includes
a handle at a proximal portion of the catheter, and
an elongated shaft extending distally along a longitudinal axis from the handle, the elongated shaft including
a treatment assembly at a distal portion of the catheter, and
a plurality of electrodes carried by the treatment assembly, the plurality of electrodes including one or more first electrodes along the distal region and one or more second electrodes along the proximal region;
increasing a first radial dimension between a distal region of the treatment assembly and the longitudinal axis to bring the first electrodes into apposition with an inner wall of the blood vessel;
increasing a second radial dimension between a proximal region of the treatment assembly and the longitudinal axis independently of movement of the distal region to bring the second electrodes into apposition with an inner wall of the blood vessel; and
activating the electrodes to modulate nerves along the blood vessel at the treatment site.

9. The method of claim 8 wherein increasing a first radial dimension occurs before increasing the second radial dimension.

10. The method of claim 8 wherein increasing the first radial dimension and increasing the second radial dimension occur simultaneously.

11. The method of claim 8 wherein increasing the first radial dimension occurs after increasing the second radial dimension.

12. The method of claim 8, further comprising decreasing the first radial dimension between the distal region of the treatment assembly and the longitudinal axis before and/or after activating the electrodes.

13. The method of claim 8, further comprising decreasing the second radial dimension between the proximal region of the treatment assembly and the longitudinal axis before, during, and/or after activating the electrodes.

14. The method of claim 8 wherein increasing the first radial dimension comprises retracting a distal end of the treatment assembly in a proximal direction while at least one of the handle and the proximal region remain fixed relative to the patient.

15. The method of claim 8 wherein increasing the second radial dimension comprises advancing a proximal end of the treatment assembly in a distal direction while at least one of the handle and the distal region remain fixed relative to the patient.

16. The method of claim 8 wherein the first radial dimension is greater than the second radial dimension.

17. The method of claim 8 wherein the first radial dimension is less than the second radial dimension.

18. The method of claim 8 wherein:
increasing the first radial dimension comprises increasing the first radial dimension to a first initial radial dimension;
increasing the second radial dimension comprises increasing the second radial dimension to a second initial radial dimension;
activating the electrodes occurs at a first time; and
wherein the method further comprises:
monitoring at least one of an electrode impedance and an electrode temperature at or at least proximate to the first and/or second electrodes at the treatment site;
based on the electrode impedance and/or the electrode temperature, increasing at least one of
the first radial dimension from the first initial radial dimension to bring at least one of the first electrodes in apposition with the inner wall of the blood vessel;
the second radial dimension from the second initial radial dimension to bring at least one of the second electrodes in apposition with the inner wall of the blood vessel; and
activating the electrodes at a second time.

19. The method of claim 8 wherein increasing the first radial dimension, increasing the second radial dimension, and activating the electrodes occurs at a first time, and wherein the method further comprises:
decreasing at least one of
the first radial dimension between the distal region of the treatment assembly and the longitudinal axis, and
the second radial dimension between the proximal region of the treatment assembly and the longitudinal axis;
repositioning the treatment assembly;
at a second time, increasing at least one of
the first radial dimension between the distal region of the treatment assembly and the longitudinal axis, and
the second radial dimension between the proximal region of the treatment assembly and the longitudinal axis; and
activating the electrodes to modulate the nerves.

20. The method of claim 19 wherein repositioning the treatment assembly includes longitudinally advancing or retracting the treatment assembly along the longitudinal axis.

21. The method of claim 19 wherein repositioning the treatment assembly includes rotating the treatment assembly about the longitudinal axis.

22. The method of claim 19 wherein repositioning the treatment assembly includes at least one of
longitudinally advancing or retracting the treatment assembly along the longitudinal axis, and
rotating the treatment assembly about the longitudinal axis.
